(19) United States
(12) Patent Application Publication
(10) Pub. No.: US 2010/0099731 A1
(43) Pub. Date: Apr. 22, 2010

(54) Use of Endothelial Interrupters in the Treatment of Neurodegenerative Diseases

(75) Inventors: Paula Grammas, Lubbock, TX (US); Randolph B. Schiffer, Las Vegas, NV (US)

Correspondence Address: Roman Aguilera III;TTUS Office of Technology Commercialization, Box 42007, Lubbock, TX, 79409-2007, US

(73) Assignee: Texas Tech University System, Lubbock, TX (US)

(21) Appl. No.: 12/571,146

(22) Filed: Sep. 30, 2009

Related U.S. Application Data
(60) Provisional application No. 61/101,886, filed on Oct. 1, 2008.

(51) Int. Cl. A61K 31/404; A61K 31/167; A61P 25/00; A61P 25/28; A61P 25/16

(52) U.S. Cl. 514/414; 514/629

(57) ABSTRACT

In various embodiments, the present invention relates generally to methods of treating at least one neurodegenerative disease by administering a medicament comprising an endothelial interrupter.

### CROSS-REFERENCE TO RELATED APPLICATIONS

This application claims the benefit under Title 35 United States Code §119(e) of U.S. Provisional Application No. 61/101,886; Filed: Oct. 1, 2008, the full disclosure of which is incorporated herein by reference.

### STATEMENT REGARDING FEDERALLY SPONSORED RESEARCH OR DEVELOPMENT

This work was funded by the National Institute for Health under grant number AG015964, AG020569, and AG028367, and the United States government may have certain rights to this invention.

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### FIELD OF THE INVENTION

The present invention relates to the use of endothelial interrupters to treat at least one neurodegenerative disease. More specifically, the present invention relates generally to methods of treating at least one neurodegenerative disease by administering a medicament comprising an endothelial interrupter.

### BACKGROUND OF THE INVENTION

Without limiting the scope of the disclosed invention, the background is described in connection with a novel approach to treating neurodegenerative diseases by administering a medicament comprising an endothelial interrupter.

Alzheimer's disease (AD) is a progressive, neurodegenerative disease that affects more than 5 million people in the United States. This number is a 10 percent increase from the previous estimate of 4.5 million and is projected to sharply increase to 8 million by 2030. It is expected that the USA alone will see some 16 million cases by 2050. At present, the few agents that are FDA-approved for treatment of AD have demonstrated only modest effects in modifying clinical symptoms for relatively short periods and none have shown a clear effect on disease progression. New therapeutic approaches are desperately needed.

Current treatments for the cognitive degeneration of AD are limited and primarily focus in two areas, including Cholinesterase inhibitors and glutamate regulation. Cholinesterase inhibitors prevent the breakdown of acetylcholine, a chemical messenger important for learning and memory. Memantine (Namenda) works by regulating the activity of glutamate, a different messenger chemical involved in learning and memory. Other approved treatments are included in Table 1 below:

TABLE 1
Approved Treatments

| Generic | Brand | Approved For | Side Effects |
|---|---|---|---|
| Donepezil | Aricept | All stages | Nausea, vomiting, loss of |
| | | | appetite and increased |
| | | | frequency of bowel |
| | | | movements. |
| Galantamine | Razadyne | Mild | Nausea, vomiting, loss of |
| | | to moderate | appetite and increased |
| | | | frequency of bowel |
| | | | movements. |
| Memantine | Namenda | Moderate | Headache, constipation. |
| | | to severe | confusion and dizziness. |
| Rivastigimine | Exelon | Mild to | Nausea, vomiting, loss of |
| | | moderate | appetite and increased |
| | | | frequency of bowel |
| | | | movements. |
| Tacrine | Cognex | Mild | Possible liver damage, |
| | | to moderate | nausea, and vomiting. |

As can be seen, these two treatment methodologies produce side effects. Likewise, none of these therapies are disease modifying therapies. Accordingly, there is a need in the art for a different treatment for neurodegenerative diseases.

### BRIEF SUMMARY OF THE INVENTION

In various embodiments, the present disclosure provides methods for treating a neurodegenerative disease. In general, treatment of a neurodegenerative disease is affected by administration of a medicament comprising an agent which interrupts endothelial activation, an endothelial interrupter.

Various embodiments disclose a method for treating a neurodegenerative disease in a patient in need thereof, the need characterized by at least one of the endothelial cell releasing at least one matrix metalloproteinase (MMP) or at least one inflammatory cytokine, the method comprising the step of: administering a medicament comprising an endothelial interrupter to the patient wherein at least one of endothelial cell release of MMP-9 or endothelial cell release of an inflammatory cytokine is reduced, the administration resulting in improved cognitive function. In an embodiment, treatment results in a level of apolipoprotein E-4 (ApoE4) in the patient's hippocampus being reduced. In various embodiments, the endothelial interrupter is a direct thrombin inhibitor.

Various further embodiments disclose methods for reducing a patient's endothelial cell release of at least one of a matrix metalloproteinase or an inflammatory cytokine, the methods comprising the step of administering a medicament comprising an endothelial interrupter to the patient.

Various further embodiments disclose a method of improving the cognitive function of a patient suffering from a neurodegenerative disease, the method comprising the step of administering a therapeutically effective amount of an endothelial interrupter to the patient.

Various further embodiments disclose a method for treating a neurodegenerative disease in a patient in need thereof, the method comprising the step of administering a medicament comprising a direct thrombin inhibitor.

The foregoing has outlined rather broadly the features of the present disclosure in order that the detailed description that follows may be better understood. Additional features and advantages of the disclosure will be described hereinafter, which form the subject of the claims.

### BRIEF DESCRIPTION OF THE OF THE DRAWINGS

For a more complete understanding of the present disclosure, and advantages thereof, reference is now made to the following descriptions to be taken in conjunction with the accompanying figures describing a specific embodiment of the disclosure, wherein:

FIG. 1 illustrates a proposed mechanism of Alzheimer's Disease;

FIG. 2 illustrates AD transgenic mice receiving daily treatments of Sunitinib malate (40 mg/kg) progressively require fewer arm entries to complete an 8 arm radial arm maze than do control untreated AD mice receiving vehicle (p<0.05). Y-axis—total number or arm entries to complete the maze. X-axis—weeks of acquisition training. The dotted line denotes perfect performance in the maze;

FIG. 3 illustrates that Sunitinib malate treatment significantly decreased release of matrix metalloproteinase-9 (MMP-9) from brain-derived endothelial cells (EC150). Nearly confluent cultures of EC150 were exposed to increasing concentration of Sunitinib malate (0-500 nM) for 4 h. Cell media were then analyzed by indirect ELISA for MMP-9. Data are mean±SEM of 3 separate experiments measured in triplicate. MMP-9 level decreases significantly (***p<0.001) compared to cells incubated without Sunitinib malate;

FIG. 4 illustrates that exposure of endothelial cells to Sunitinib malate simultaneously with menadione significantly decreased release of MMP-9 (*p<0.05 compared to menadione treatment alone). Endothelial cells were exposed for 4 h to 500 nM Sunitinib malate prior to (Pre), simultaneously (Co), or after (Post) 4 h menadione treatment (25 μM). Cell media were then analyzed by indirect ELISA for MMP-9. Data are mean±SEM of 3 separate experiments measured in triplicate. (**p<0.01, ***p<0.001 compared to untreated cells);

FIG. 5 illustrates that exposure of endothelial cells to Sunitinib malate decreased menadione-mediated release of TNF-α. Endothelial cells were exposed for 4 h to 500 nM Sunitinib malate prior to (Pre), simultaneously (Co), or after (Post) 4 h menadione treatment (25 μM). Cell media were then analyzed by indirect ELISA for TNF-α. Data are mean±SEM of 3 separate experiments measured in triplicate. (**p<0.01, ***p<0.001 compared to menadione treatment alone);

FIG. 6 illustrates that rat brain endothelial cells were incubated with increasing concentration of acetaminophen (APAP) and then oxidatively challenged with 25 μM menadione for 2-3 h. Cell viability was determined with MTT assay. Number of viable cells in untreated cells (control) was defined as 100%. Data are mean±SD of 3 separate experiments. Cell survival increases significantly (***p<0.001) compared to cells incubated without acetaminophen;

FIG. 7 illustrates that expression of Macrophage Inflammatory proteins (MIP-1α) released into the supernatant in brain endothelial cells pretreated with acetaminophen (APAP) and incubated with 25 μM of menadione, was detected by ELISA. Data are mean±SD values from 3 separate experiments. **p<0.01, ***p<0.001 vs. menadione without APAP;

FIG. 8 illustrates that brain endothelial cells were pretreated with acetaminophen (APAP) and incubated with 25 μM of menadione for 2-3 h. Total RNA extracted from the above treated cells was transcribed with Bcl₂ specific primers. PCR amplified products of Bcl₂ were demonstrable as discrete bands at 331 bp. The housekeeping gene actin confirms equal amount of total RNA (lower panel) used for RT-PCR. Representative gel shown from 3 separate experiments depicting Bcl₂ protein expression with acetaminophen.

### DETAILED DESCRIPTION OF THE INVENTION

Disclosed herein is a novel approach to treating neurodegenerative diseases by administering a medicament comprising an endothelial interrupter. The numerous innovative teachings of the present invention will be described with particular reference to several embodiments (by way of example, and not of limitation).

In the following description, certain details are set forth such as specific quantities, sizes, etc. so as to provide a thorough understanding of the present embodiments disclosed herein. However, it will be obvious to those skilled in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

The following definitions and explanations are meant and intended to be controlling in any future construction unless clearly and unambiguously modified in the following Description or when application of the meaning renders any construction meaningless or essentially meaningless. In cases where the construction of the term would render it meaningless or essentially meaningless, the definition should be taken from Webster's Dictionary, 3rd Edition. Definitions and/or interpretations should not be incorporated from other patent applications, patents, or publications, related or not, unless specifically stated in this specification or if the incorporation is necessary for maintaining validity.

As used herein, the term “Alzheimer's Disease” (“AD”) means and refers to a progressive form of presenile dementia that is similar to senile dementia. It is common that some of the first symptoms of AD are impaired memory which is followed by impaired thought and speech and can result in complete helplessness.

As used herein, Argatroban is an anticoagulant that is a small molecule direct thrombin inhibitor.

As used herein, Bivalirudin (Angiomax) is a drug that belongs to the anticoagulant class and acts as a direct thrombin inhibitor. Chemically it constitutes a synthetic congener of the naturally occurring drug hirudin. Bivalirudin directly inhibit thrombin by specifically binding as well to the catalytic site and to the anion-binding exosite of circulating and clot- or thrombus-bound thrombin. Thrombin is a serine protease that plays a central role in the thrombotic process, acting to cleave fibrinogen into fibrin monomers and to activate Factor XIII to Factor XIIIa, allowing fibrin to develop a covalently cross-linked framework which stabilizes the thrombus; thrombin also activates Factors V and VIII, promoting further thrombin generation, and activates platelets, stimulating aggregation and granule release. It is a reversible inhibitor of thrombin.

As used herein, the term “cognitive function” means and refers to at least one of memory, perception, orientation, reasoning, and/or judgment.

As used herein, the term “endothelial activation” means and refers to at least one physiological change in endothelial cells that leads to induction of biosynthetic properties that contribute to angiogenesis, inflammatory response, cellular adhesion properties, and immunological responses.

As used herein, the term “neurodegenerative disease” means and refers to a disorder caused by the deterioration of certain nerve cells (neurons). Changes in these cells cause them to function abnormally, eventually bringing about their death or degeneration. Examples of such diseases include, but are not limited to Alzheimer's, Parkinson's, and Creutzfeldt-Jakob, multiple sclerosis, cerebral ischemias, epilepsy, neurodegenerative disease caused by traumatic injury, and/or the like.

As used herein, Acetaminophen, 4′-hydroxyacetanilide, is a nonopiate, non-salicylate analgesic and antipyretic which occurs as a white, odorless, crystalline powder, possessing a slightly bitter taste. Its structure is as follows:

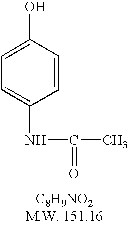

Acetaminophen is used to relieve mild to moderate pain from headaches, muscle aches, menstrual periods, colds and sore throats, toothaches, backaches, reactions to vaccinations (shots), to reduce fever, and/or the like. Acetaminophen may also be used to relieve the pain of osteoarthritis (arthritis caused by the breakdown of the lining of the joints). Acetaminophen is in a class of medications called analgesics (pain relievers) and antipyretics (fever reducers). It works by changing the way the body senses pain and by cooling the body.

As used herein, Sunitinib malate, a Butanedioic acid, hydroxy-, (2S)-, compound with N[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide (1:1), is an oral, small-molecule of the class of 2-O Sulfatase, multi-targeted receptor tyrosine kinase (RTK) inhibitor that was approved by the FDA for the treatment of renal cell carcinoma (RCC) and imatinib-resistant gastrointestinal stromal tumor (GIST) on Jan. 26, 2006. It has a molecular formula is C22H27FN4O2.C4H6O5 and the molecular weight is 532.6 Daltons. Sunitinib malate inhibits cellular signaling by targeting multiple RTKs. These include all platelet-derived growth factor receptors (PDGF-R) and vascular endothelial growth factor receptors (VEGF-R), which play a role in both tumor angiogenesis and tumor cell proliferation. The simultaneous inhibition of these targets therefore leads to both reduced tumor vascularization and cancer cell death, and ultimately tumor shrinkage. Sunitinib also inhibits KIT (CD117), the RTK that drives the majority of GISTs. In addition, sunitinib inhibits other RTKs including RET, CSF-1R, fit3, and others.

As used herein, the term “thrombus” means and refers to a fibrinous clot formed in a blood vessel or in a chamber of the heart.

As used herein, the term “blocker”, “inhibitor”, “interrupter” or “antagonist” means a substance that retards or prevents a chemical or physiological reaction or response. Common blockers or inhibitors include but are not limited to antisense molecules, antibodies, antagonists and their derivatives.

As used herein, the term “patient” means and refers to a human or animal.

As used herein, the term “pharmaceutically acceptable salts” means and refers to compounds according to the invention used in the form of salts derived from inorganic or organic acids and bases.

Included among acid salts, for example, are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectianate, persulfate, phenylproprionate, picrate, pivalate, propionate, succinate, tartrate, thiocyanate, tosylate and undecanoate.

Salts derived from appropriate bases include alkali metal (e.g. sodium), alkaline earth metal (e.g., magnesium), ammonium and NW₄₊ (wherein W is C₁₋₄ alkyl). Physiologically acceptable salts of a hydrogen atom or an amino group include salts or organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids. Physiologically acceptable salts of a compound with a hydroxy group include the anion of the compound in combination with a suitable cation such as Na₊, NH₄₊, and NW₄₊ (wherein W is a C₁₋₄ alkyl group).

Pharmaceutically acceptable salts include salts of organic carboxylic acids such as ascorbic, acetic, citric, lactic, tartaric, malic, maleic, isothionic, lactobionic, p-aminobenzoic and succinic acids; organic sulphonic acids such as methanesulphonic, ethanesulphonic, benzenesulphonic and p-toluenesulphonic acids and inorganic acids such as hydrochloric, sulphuric, phosphoric, sulphamic and pyrophosphoric acids.

For therapeutic use, salts of the compounds according to the invention will be pharmaceutically acceptable. However, salts of acids and bases that are not pharmaceutically acceptable may also find use, for example, in the preparation or purification of a pharmaceutically acceptable compound.

Preferred salts include salts formed from hydrochloric, sulfuric, acetic, succinic, citric and ascorbic acids.

As used herein, the term “chemically feasible” refers to a connectivity of atoms such that the chemical valency of each atom is satisfied. For example, an oxygen atom with two bonds and a carbon atom with four bonds are chemically feasible.

As used herein, the term “Lepirudin” (Refludan) is an anticoagulant which functions as a direct thrombin inhibitor.

As used herein, Ximelagatran is an anticoagulant which functions as a direct thrombin inhibitor.

As used herein, “therapeutically effective amount” means and refers to that amount of the compound being administered which will relieve to some extent one or more of the symptoms of the disorder being treated. In the case of a neurodegenerative disease, a therapeutically effective amount is capable of improving the cognitive function of the patient in various embodiments.

As used herein, Desirudin is an anticoagulant (blood thinner) and a direct thrombin inhibitor.

Referring to the drawings and tables in general, it will be understood that the illustrations are for the purpose of describing a particular embodiment of the disclosure and are not intended to be limiting thereto.

In various embodiments, the present invention relates generally to methods of treating at least one neurodegenerative disease by administering a medicament comprising an endothelial interrupter. Particularly included endothelial interrupters include, but are not limited to Bivalirudin (Angiomax), Lepirudin (Refludan), Argatroban (Argatroban), Ximelagatran (Exanta), Desirudin (Iprivask), and/or the like. In various embodiments, the endothelial interrupters are direct thrombin inhibitors.

In addition to the compounds of this invention, pharmaceutically acceptable salts of the compounds of this invention may also be employed in compositions to treat or prevent neurodegenerative diseases.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium and potassium), alkaline earth metal (e.g., magnesium), ammonium and N—(C₁₋₄ alkyl)4+salts. This invention also envisions the quaternization of any basic nitrogen-containing groups of the compounds disclosed herein. Water or oil-soluble or dispersible products may be obtained by such quaternization.

Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, aqueous suspensions or solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of inhibitor that may be combined with the carrier materials to produce a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of inhibitor will also depend upon the particular compound in the composition.

Depending upon the particular neurodegenerative disease condition to be treated or prevented, additional drugs, which are normally administered to treat or prevent that condition may be administered together with the inhibitors of this invention. For example, chemotherapeutic agents or other anti-proliferative agents may be combined with the endothelial interrupters of this invention to treat proliferative diseases.

Those additional agents may be administered separately, as part of a multiple dosage regimen, from the endothelial interrupter-containing composition. Alternatively, those agents may be part of a single dosage form, mixed together with the endothelial interrupter in a single composition.

The invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes to the claims that come within the meaning and range of equivalency of the claims are to be embraced within their scope. Further, all published documents, patents, and applications mentioned herein are hereby incorporated by reference, as if presented in their entirety.

### EXAMPLES

Work from our laboratory has led us to conclude that brain blood vessels are dysfunctional in neurodegenerative diseases, including AD, and contribute to disease pathogenesis. We believe that in neurodegenerative diseases an abnormal pathologically altered brain endothelium produces factors that are toxic to neurons and that the cerebral vasculature is an important mediator of neuronal injury in neurodegenerative diseases such as AD. We have shown that AD microvessels express and release numerous inflammatory mediators, including thrombin, tumor necrosis factor alpha (TNFα), tumor growth factor beta (TGFβ), interleukin-6 (IL-6), interleukin-8 (IL-8), nitric oxide (NO), and MCP-1, all of which have been implicated in vascular activation/angiogenesis. Part of this data is presented below. Taken together, these data suggest a heretofore-unexplored connection between vascular activation/angiogenesis and AD and have led us to propose the hypothesis that vascular activation contributes to AD pathology and cognitive impairment.

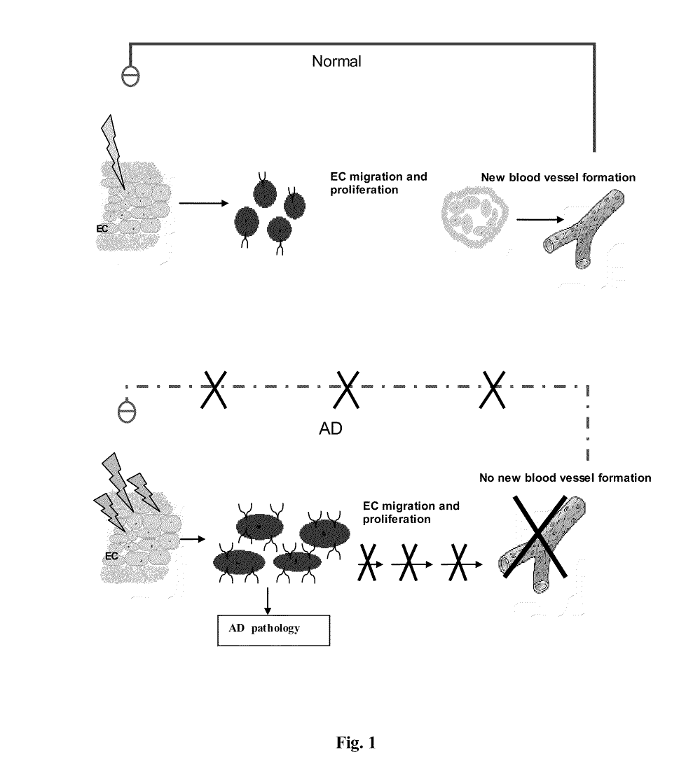

Despite increases in several pro-angiogenic factors in the AD brain, evidence for increased vascularity in AD is lacking. On the contrary, it has been suggested that the angiogenic process is delayed and/or impaired in aged tissues, with several studies showing decreased microvascular density in the AD brain. This suggests that an imbalance between pro- and anti-angiogeneic processes occurs in the AD brain. In our working model, we hypothesize that in response to a persistent stimulus, such as cerebral hypoperfusion, one of the major clinical features in AD, brain endothelial cells become activated. Despite the continued presence of the stimulus, an imbalance of pro- and anti-angiogenic factors or aborted angiogenic signaling prevents new vessel growth. Therefore, in the absence of feedback signals to shut off vascular activation, endothelial cells remain activated and elaborate a large number of proteases, inflammatory proteins and other gene products with biologic activity that could injure or kill neurons. A diagram of the hypothesis is shown in FIG. 1. A diagram of hypothesis. ( [symbol] )=stimuli (for example, and not by way of limitation, hypoxia, IL-1β); ( [symbol] )=proteases, inflammatory proteins and other gene products with biologic activity; (⊖) feedback inhibition.

Brain Blood Vessels Express Inflammatory Mediators Associated with Vascular Activation/Angiogenesis.

Inflammatory factors are elevated in brain microvessels in AD. A large number of inflammatory mediators have been documented in pathologically vulnerable areas of the AD brain. The objective of this study was to compare both the release and presence of microvessel, associated cytokines in vessels isolated from the brains of AD patients and age-matched controls. Our results demonstrated that unstimulated AD vessels release significantly higher levels, assessed by ELISA, of IL-1β, IL-6 and TNFα compared to non-AD microvessels. Western blot analysis of microvessel-associated proteins showed that MCP-1 and IL-1β were highly expressed in AD vessels but not detected in controls (15) (Reference, item # 1). These results suggest that the cerebral microcirculation contributes inflammatory mediators to the milieu of the AD brain and may be involved in the pathogenesis of neuronal injury and death in this disorder. Furthermore the cytokines over expressed by AD microvessels are key regulators of vascular activation/angiogenesis.

Cerebrovascular TGF-β contributes to inflammation in the AD brain. TGF-β plays a central role in the response of the brain to injury and is increased in the brain in AD. In this study, we determined whether expression of TGF-β was abnormal in the microvasculature in AD and whether TGF-β affected vascular production of pro-inflammatory cytokines, IL-1β and TNFα. Results from western blot analysis and ELISA indicated a higher level of TGF-β in AD vessels compared to controls. To determine whether TGF-β affects vascular release of inflammatory factors, cultured brain endothelial cells were treated with TGF-β and levels of IL-1β and TNFα determined. Both ELISA and western blot analyses showed that untreated endothelial cells express little IL-1β or TNFα, but incubation with TGF-β resulted in robust endothelial expression of these factors (Reference, item # 2). Studies have shown that TGF-β mediated signaling is critical to controlling the “angiogenic switch”. Thus, our results documenting increased vessel-derived TGF-β in AD suggest angiogenic vascular changes in this disease.

Nitric oxide synthase is elevated in brain microvessels in Alzheimer's disease. In this study, we compared the nitric oxide synthase (NOS) activity of brain microvessels isolated from AD and control brains. L-[³H]-citrulline, the stable co-product generated with NO from L-[³H]-arginine, was measured as an indicator of NOS activity. The results indicated a significant increase in NOS activity in microvessels isolated from Alzheimer brains. In addition, using antibodies to both the endothelial (eNOS) and inducible (iNOS) isoforms, we demonstrated a significant increase in both isoforms, which was especially large for iNOS, in AD-derived vessels. Elevated vascular production of NO, a potentially neurotoxic mediator in the CNS, may contribute to the susceptibility of neurons to injury and cell death in AD. Also, NO is a complex mediator of angiogenesis, showing both pro- and anti-angiogenic properties (Reference, item # 3).

Angiogenic proteins are expressed by brain blood vessels in AD. Data are emerging to support the idea that mediators of angiogenesis are found in the AD brain. The objective of this study is to compare the expression of the angiogenic mediator's vascular endothelial growth factor (VEGF), angiopoietin, and matrix metalloproteinases (MMPs) in the microcirculation of AD patients and age-matched controls. Our results indicate that angiopoietin-2 and VEGF are expressed by AD—but not control-derived microvessels. AD-derived microvessels also release higher levels of MMP-2 and MMP-9 compared to controls. The data show that despite high levels of MMP-9, assessed by western blot, MMP-9 activity is not detectable in AD microvessels. In this regard we find high levels of the tissue inhibitor of matrix metalloproteinases-1 (TIMP-1) in AD, but not control vessels. Furthermore, we explore the ability of thrombin, previously shown to be present in AD microvessels, to affect TIMP expression in cultured brain endothelial cells and find that thrombin causes up regulation of TIMP-1. These data show that angiogenic changes occur in the microcirculation of the AD brain and suggest that if these changes are contributory to disease pathogenesis, targeting the abnormal brain endothelial cell would provide a novel therapeutic approach for the treatment of this disease (Reference, item # 4).

Thrombin a Key Regulator of Inflammation, Angiogenesis and Neurotoxicity in AD.

Thrombin and inflammatory proteins are elevated in Alzheimer's disease microvessels: Implications for disease pathogenesis. The notion that microvascular abnormalities contribute to deleterious changes in the AD brain is supported by work from our laboratory and others demonstrating biochemical and functional alterations of the microcirculation in AD. The objective of this study is to determine whether levels of neurotoxic (thrombin) and inflammatory proteins (IL-8; integrins αᵥβ₃ and αᵥβ₅) are altered in microvessels isolated from AD patients compared to levels in vessels obtained from non-demented age-matched controls. We also evaluate expression, in AD and control microvessels, of the transcription factor hypoxiainducible factor 1-α (HIF1-α), which regulates pro-inflammatory gene expression, and the regulation of HIF1-α expression by thrombin in cultured brain endothelial cells. Our results indicate that in AD there are high levels of expression of the neurotoxic protease thrombin and the inflammation-associated proteins IL-8 and αᵥβ₃ and αᵥβ₅ integrins. HIF1-alpha is higher in AD microvessels compared to control and thrombin treatment of cultures brain endothelial cells results in increased expression of HIF1-alpha. These data suggest that in AD the cerebral microcirculation is a source of neurotoxic and inflammatory mediators and as such contributory to pathologic processes ongoing in the AD brain (Reference, item # 5).

Neurotoxicity of thrombin. Thrombin accumulation has been documented in the senile plaques of the Alzheimer brain. In addition, activation or over-expression of the protease-activated thrombin receptor (PAR-1) has been shown to induce motor neuron degeneration. In our study, we examined both thrombin-evoked apoptosis and necrosis in the same well, where comparing and contrasting these two processes is most valid, we found a significant apoptotic effect and little effect on necrosis in cerebral cortical cultures or differentiated PCl2 cells (Reference, item # 6). In primary cortical cell cultures and differentiated PCl2 cells thrombin, of the mediators tested, evoked the highest level of nucleosome accumulation, suggesting that this protein may be an important mediator of neuronal cell death.

Injured endothelial cells release thrombin and generate neurotoxic apolipoprotein E fragments. The objective of this study was to determine if injured brain endothelial cells could be a source of thrombin and neurotoxic apoE fragments. Brain endothelial cells were injured by oxidant and/or inflammatory stress. This was accomplished by incubating cultured endothelial cells with either, sodium nitroprusside (SNP), inflammatory cocktail (LPS, IL-113, IL-6, IFN-γ, TNFα) or both for 24 h. The conditioned medium was then (a) analyzed for thrombin using a chromogenic thrombin activity assay, (b) incubated with apoE4 for 24 h and then analyzed by western blot for detection of apoE4 fragments, or (c) incubated with apoE4 and added to neuronal cultures for 24 h. The results showed that treatment of endothelial cells with either SNP or the inflammatory cocktail caused a significant (p<0.005) increase in the release of thrombin compared to untreated control cultures. Also, endothelial cell conditioned media generated several apoE4 fragments (20-29 kDa). This pattern was consistent with that observed using purified thrombin and was inhibited by the thrombin inhibitor hiruidin. Finally, addition of SNP— or inflammatory cocktail-treated endothelial cell conditioned medium to neuronal cell cultures resulted in an increase in neuronal cell death, assessed by nucleosome ELISA, compared to uninjured endothelial cell conditioned medium. These data suggest that endothelial cell injury results in thrombin release and that a damaged microvasculature brain could be a source of neurotoxic factors (Reference, item # 7).

Thrombin, a mediator of neurotoxicity and memory impairment. Thrombin has been found in neuritic plaques in AD. Also, traumatic brain injury, where neurons are exposed to high thrombin levels is associated with an increased incidence of AD. Our objective was to determine the effects of thrombin administered in vivo on cognitive function and neuropathology. Rats were trained using a radial eight-arm maze and then thrombin (25 or 100 nM, 0.25 μg/h, 28 days) or vehicle was delivered via intra cerebroventricular infusion. Animals that received 100 nM thrombin demonstrated cognitive impairments including deficits in reference memory and an increase in task latency. Also, significant neuropathology was detected in these animals such as enlargement of cerebral ventricles, an increased number of TUNEL-positive cells, astrogliosis, and an increase in the immunoreactivity for phosphorylated neurofilament, and apolipoprotein-E fragments. Thrombin-induced changes in cognitive function and ventricular enlargement were inhibited by hirudin. These findings demonstrate that thrombin is a mediator of neurotoxicity and cognitive deficits and suggest that inhibition of thrombin may be a treatment strategy for AD- or head trauma associated cognitive deficits (Reference, item # 8).

Chronic thrombin exposure results in an increase in apolipoprotein-E levels. Studies have shown that individuals with both a history of traumatic brain injury and inheritance of apolipoprotein E-4 (ApoE4) allele are associated with a poor neurologic outcome and an increased risk for Alzheimer's disease. We assessed the hypothesis that thrombin released during brain injury causes an increase in apolipoprotein-E levels and such increase in the levels of apolipoprotein-E4 isoform may have amyloidogenic effects. Rats received either thrombin (100 nM, 0.25 microl/hr, 28 days) or vehicle via intracerebroventricular infusion. Thrombin treatment increased apolipoprotein-E levels in hippocampus as compared to vehicle treatment (p<0.001). Infusion of human apolipoprotein-E4 (0.6 ng/hr, i.c.v., 56 days) into rats resulted in beta-amyloid deposition and increased the number of GFAP-positive astrocytes. ApoE4 infusion also resulted in significant spatial memory deficits. These findings suggest that thrombin released during brain injury may contribute to an increase in apolipoprotein-E levels. Such increase in Apolipoprotein-E4 isoform facilitates beta-amyloid deposition and cognitive deficits (Reference, item # 9).

Thrombin and oxidative stress cause induction of cell cycle proteins and cell death in cultural neurons. Recent studies provide evidence that cell cycle proteins playa critical role as mediators of programmed cell death, both in the developing nervous system, and as a result of inappropriate activation during the course of certain neuropathological conditions. CyclinD1, an early marker of the G1 transition, has been implicated as a regulator of neuronal apoptosis. We have previously shown that thrombin and oxidative stress cause neuronal cell death. The objective of this study is to determine whether thrombin or oxidative stress alter expression of cell cycle proteins. Rat neuronal cortical cell cultures were treated with either thrombin (100 nM) or sodium nitroprusside (SNP, 10 μM), a nitric oxide donor, for 24 h. Neuronal cell cultures were then analyzed for cyclin D1 mRNA expression, using RT-PCR analysis, and neuronal apoptosis, quantified by nucleosome ELISA. Our results show that both thrombin and oxidative injury cause an increase in cyclin D1 expression in cultured neurons and evoke neuronal cell death. These data suggest that activation of the cell cycle in neurons may contribute to neuronal cell death in conditions, such as AD, where thrombin and oxidative stress have been implicated (Reference, item # 10).

Inhibiting Vascular Activation/Inflammation Improves Cognition in AD Mice.

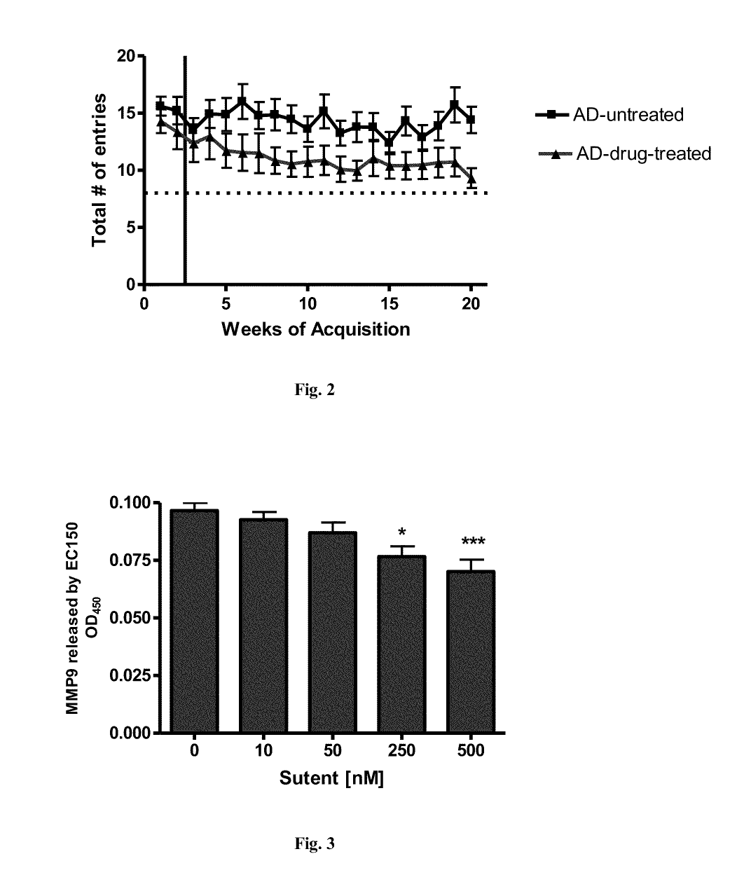

Preliminary experiments using the AD transgenic mouse APPSWE 2576 demonstrate that administration of the endothelial interrupter Sunitinib malate (Su 11248, sunitinib malate) appears to improve cognitive performance. Animals are trained on a radial arm maze for 14 days. Experimental animals then receive Sunitinib malate (40 mg/kg) daily in their diet and are retested on the maze. As indicated in FIG. 2, animals that receive Sunitinib malate perform significantly (p<0.05) better than AD mice not receiving the drug.

In Vitro Effects of Endothelial Interrupter on Brain Endothelial Cells.

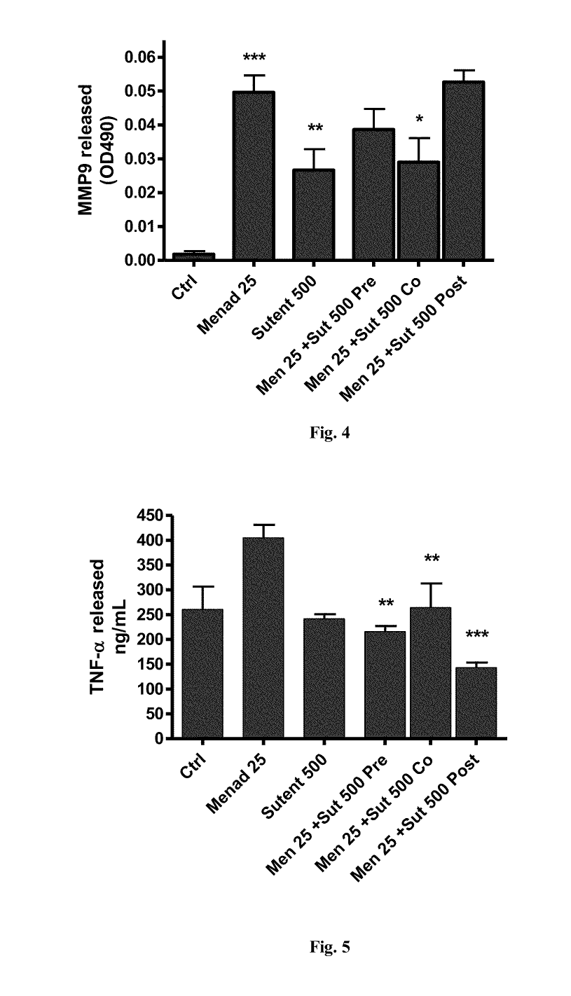

Based on the results from our experiments in AD mice showing that administration of an endothelial interrupter improved cognitive performance, we examined the direct effect of Sunitinib malate on brain endothelial cells in culture. Treatment of cultured brain endothelial cells with Sunitinib malate (0-500 nM) for 4 h significantly decreased release of matrix metalloproteinase-9 (MMP-9), an important angiogenic protein and neurotoxin (FIG. 3). In addition, exposure of brain endothelial cells to the oxidant stressor menadione causes increased release of both MMP-9 and the inflammatory cytokine TNFα. Exposure of endothelial cells to Sunitinib malate, either prior to menadione or simultaneously resulted in decreased release of both MMP-9 (FIG. 4) and TNFα (FIG. 5).

Treatment of AD mice with antiangiogenesis compounds. Various angiogenesis inhibitors, or endothelial interrupters, tested include Sorafenib tosylate (Nexavar), Sunitinib (Sunitinib malate), and Lenalidomide (Revlimid). Each agent is capable of penetrating the central nervous system, and has been clinically tested for safety and biological activity.

All agents are orally administered and bioavailable. Sorafenib tosylate (Nexavar) a small molecule, multi-kinase inhibitor directed against the VEGF receptor and Raf kinase. Sunitinib (Sunitinib malate) is a novel small molecule receptor tyrosine kinase inhibitor. Lenalidomide (Revlimid) is a small molecule of analog of thalidomide, with antiangiogenic and anti-cytokine properties. The AD mice models begin receiving the drug at 2 months of age. Animals at this time are behaviorally and neuropathologically normal. All drugs are dosed using the allometric scaling method of C Sedgwick, based on known optimal biological doses in mice; Sorafenib tosylate (60 mg/kg/day), or Sunitinib (40 mg/kg/day), or Lenalidomide (100 mg/kg/day). Control animals receive a placebo.

Thrombin: vascular-derived mediator of neuronal injury. Based on several of our studies we hypothesize that anti-thrombin drugs could be effective AD and other cognitive disorder therapeutics.

Direct Thrombin Inhibitors:

There are a variety of direct thrombin inhibitors. Examples include, but are not limited to, Bivalirudin (Angiomax), Lepirudin (Refludan), Argatroban (Argatroban), Ximelagatran (Exanta), Desirudin (Iprivask), and/or the like. Direct thrombin inhibitors (DTIs) are a new class of anticoagulants that bind directly to thrombin and block or interfere with its interaction with its substrates. Certain DTIs, such as recombinant hirudins, bivalirudin, and ximelagatran, either alone or in combination with melagatran, have undergone extensive evaluation in phase 3 trials for the prevention and treatment of arterial and venous thrombosis. The evidence concerning the clinical applicability of other DTIs, such as argatroban and dabigatran, is limited to phase 2 studies. Four parenteral DTIs have been approved by the Food and Drug Administration (FDA) in North America: hirudin and argatroban for the treatment of heparin-induced thrombocytopenia, bivalirudin as an alternative to heparin in percutaneous coronary intervention, and desirudin as a prophylaxis against venous thromboembolism in hip replacement.

Mechanisms of Action

Coagulation Cascade and Generation of Thrombin

After injury to a vessel wall, tissue factor is exposed on the surface of the damaged endothelium. The interaction of tissue factor with plasma factor VII activates the coagulation cascade, producing thrombin by stepwise activation of a series of proenzymes. Thrombin is central in the clotting process: it converts soluble fibrinogen to fibrin; activates factors V, VIII, and XI, which generates more thrombin; and stimulates platelets. Furthermore, by activating factor XIII, thrombin favors the formation of cross-linked bonds among the fibrin molecules, stabilizing the clot. The coagulation cascade is regulated by natural anticoagulants, such as tissue factor pathway inhibitor, the protein C and protein S system, and antithrombin, all of which help to restrict the formation of the hemostatic plug to the site of injury.

Differences from Heparins

Thrombin-inhibiting drugs can block the action of thrombin by binding to three domains: the active site or catalytic site and two exosites. Located next to the active site, exosite 1 acts as a dock for substrates such as fibrin, thereby orienting the appropriate peptide bonds in the active site. Exosite 2 serves as the heparin-binding domain. Thrombin is inhibited indirectly by low molecular-weight heparins, because these drugs strongly catalyze the function of antithrombin. A heparin-thrombin-antithrombin complex is formed in which heparin binds simultaneously to exosite 2 in thrombin and to antithrombin. Furthermore, heparin can act as a bridge between thrombin and fibrin by binding both to fibrin and exosite 2. Because both thrombin exosites are occupied within this fibrin-heparin-thrombin complex, the enzymatic activity of thrombin is relatively protected from inactivation by the heparin-antithrombin complex. Thus, heparins have a reduced capacity for the inhibition of fibrin-bound thrombin, which appears to be detrimental, because active thrombin further triggers thrombus growth.

Since DTIs act independently of antithrombin, they can inhibit thrombin bound to fibrin or fibrin degradation products. Bivalent DTIs block thrombin at both the active site and exosite 1, whereas univalent DTIs bind only to the active site. The group of bivalent DTIs includes hirudin and bivalirudin, whereas argatroban, melagatran (and its oral precursor, ximelagatran), and dabigatran are univalent DTIs). Native hirudin and recombinant hirudins (lepirudin and desirudin) form an irreversible 1:1 stoichiometric complex with thrombin. In a similar way, bivalirudin, a synthetic hirudin, binds to the active site and exosite 1, but once bound, it is cleaved by thrombin, thereby restoring the active-site functions of thrombin. Therefore, in contrast to the hirudins, bivalirudin produces only a transient inhibition of thrombin.

By interacting only with the active site, univalent DTIs inactivate fibrin-bound thrombin. Argatroban and melagatran (like bivalirudin) dissociate from thrombin, leaving a small amount of free, enzymatically active thrombin available for hemostatic interactions.

By reducing the thrombin-mediated activation of platelets, DTIs also have an antiplatelet effect. Since DTIs do not bind to plasma proteins, these agents should produce a more predictable response than does unfractionated heparin and should be more effective than low molecular-weight heparins because they inhibit fibrin-bound thrombin.

Acetaminophen Dampens Activation of Brain Endothelial Cells.

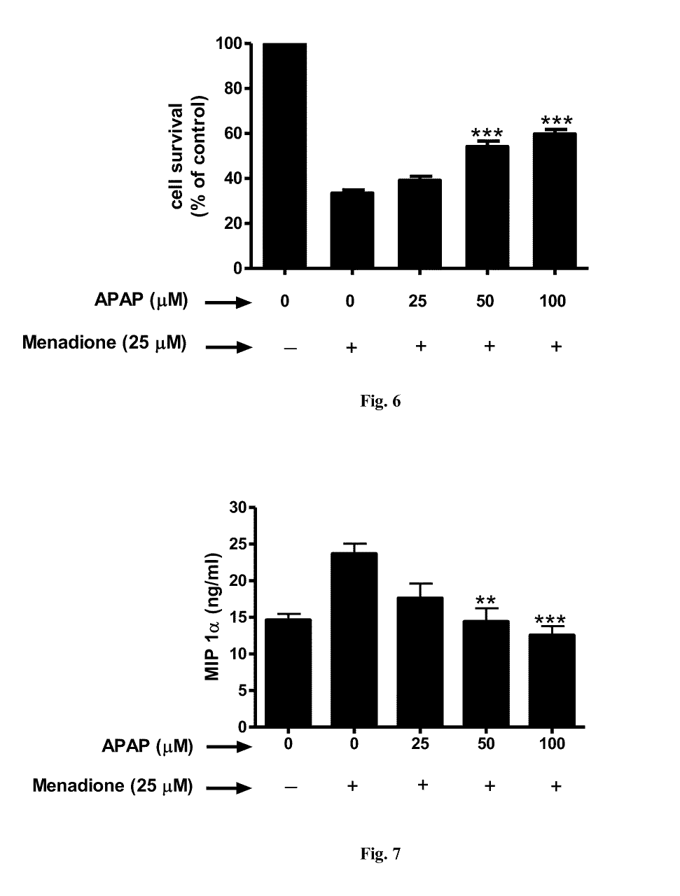

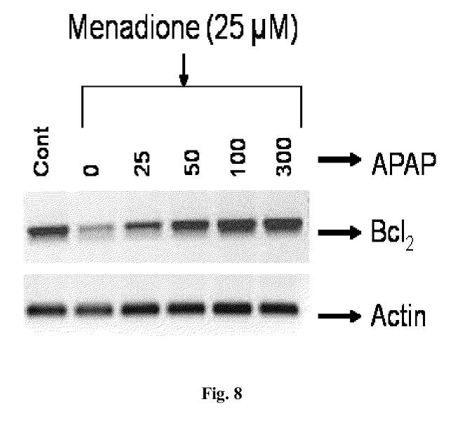

Numerous studies have suggested a link between oxidative stress and vascular inflammation. We found that pretreatment of cultured endothelial cells with acetaminophen caused a significant increase in cell survival when cells were subsequently exposed to the oxidant stressor menadione (FIG. 6). Also, treatment of cultured endothelial cells with menadione caused release of several inflammatory proteins including RANTES, MIP1α, TNFα, IL-1β. We showed that pretreatment of endothelial cells with acetaminophen decreased expression of these inflammatory proteins; data for MIP1α are shown in FIG. 7. Part of the protective effect of acetaminophen on brain endothelial cells appears to be due to induction of the anti-apoptotic protein Bcl2 (FIG. 8). Acetaminophen is a safe and effective analgesic/antipyretic drug at therapeutic doses, although its hepatotoxicity at high doses has been extensively studied and documented. In contrast, few studies have explored a possible cerebroprotective effect, despite increasing evidence that acetaminophen has anti-inflammatory and anti-oxidant properties. Our in vitro studies on brain endothelial cells taken together with in vitro studies on neurons and in vivo work showing improved cognition with acetaminophen suggest a heretofore unappreciated therapeutic potential for this drug in neurodegenerative diseases such as Alzheimer's disease that are characterized by oxidant and inflammatory stress.

The disclosed invention is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms have been defined. Terms defined herein have meanings as commonly understood by a person of ordinary skill in the areas relevant to the present invention. Terms such as “a”, “an”, and “the” are not intended to refer to only a singular entity, but include the general class of which a specific example may be used for illustration. The terminology herein is used to describe specific embodiments of the invention, but their usage does not delimit the disclosed invention, except as may be outlined in the claims.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of those skilled in the art to which this invention pertains. All publications and patent application are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of and “consisting essentially of,” respectively, shall be closed or semi-closed transitional phrases.

### REFERENCES

1. Grammas P, Ovase R. Inflammatory factors are elevated in brain microvessels in Alzheimer's disease. Neurobiol Aging, 22:837-842, 2001.
2. Grammas P, Ovase R. Cerebrovascular TGF-β contributes to inflammation in the Alzheimer's brain. Am J Pathol, 160:1583-1587, 2002.
3. Dorheim M A, Tracey W R, Pollock J S, Grammas P. Nitric oxide is elevated in Alzheimer's brain microvessels. Biochem Biophys Res Comm, 205:659-665, 1994.
4. Thirumangakudi L, Ghatreh-Samany P, Owoso A, Wiskar B, Grammas P. Angiogenic proteins are expressed by brain blood vessels in Alzheimer's disease. J Alz Dis 10: 111-118, 2006.
5. Grammas P, Ghatreh-Samany P, Thirmangalakudi L. Thrombin and Inflammatory proteins are elevated in Alzheimer's disease microvessels: Implications for disease pathogenesis. J Alz Dis, 9: 51-58, 2006.
6. Reimann-Philips U, Ovase R, Lapus M, Weigel P H, Grammas P. Mechanisms of cell death in primary cortical neurons and PC12 cells. J Neurosci Res, 64:654-660, 2001.
7. Grammas P, Ottman T, Reimann-Phillip U, Larabee J, Weigel P H. Injured endothelial cells release neurotoxic thrombin. J Alz Dis, 6: 275-281, 2004.
8. Mhatre M, Nguyen A, Pham T, Adesina A, Grammas P. Thrombin, a mediator of neurotoxicity and memory impairment. Neurobiol Aging, 25:783-793, 2004.
9. Mhatre M, Hensley K, Nguyen A, Grammas P. Chronic thrombin exposure results in an increase in apolipoprotein-E levels. J Neurosci Res, 84:444-449, 2006.
10. Vittal-Rao H, Thirumangakudi L, Desmond P, Grammas P. Cyckin D1, Cdk4 and Bim mediate thrombin-induced apoptosis in cultured cortical neurons. J Neurochem, 10: 498-505, 2007.
   First time publications where hypothesis is specifically presented:
1. Neuwalt E, Abbott N J, Abrey L, Banks, W A, Blakley B, Davis T, Engelhardt B, Grammas P, Nedergaard M, Nutt J, Pardridge W, Rosenberg G A, Smith Q, Drewes LR. Strategies to advance translational research into brain barriers. Lancet Neurol, 7:84-96, 2008.
2. Grammas P, Nutt j, Johanson C, Stopa E, Quinn J, Maron L, Sah D, Wu J, Leenders N, Zlokovic B. Nuerodegeneration and the Brain Barriers. http://www.ibbsocc.org/IBBS/Reports.htm, 2008.

I claim:

1. A method for treating a neurodegenerative disease in a patient in need thereof, said need characterized by at least one of said endothelial cell releasing at least one matrix metalloproteinase (MMP) or at least one inflammatory cytokine, said method comprising the step of
administering a medicament comprising an endothelial interrupter to said patient wherein at least one of endothelial cell release of MMP-9 or endothelial cell release of an inflammatory cytokine is reduced, said administration resulting in improved cognitive function.

2. The method of claim 1, wherein a level of apolipoprotein E-4 (ApoE4) in said patient's hippocampus is reduced.

3. The method of claim 1, wherein said medicament is administered orally, parenterally, by inhalation spray, topically, rectally, nasally, buccally, vaginally or via an implanted reservoir.

4. The method of claim 1, wherein the endothelial interrupter is a direct thrombin inhibitor.

5. A method for reducing a patient's endothelial cell release of at least one of a matrix metalloproteinase or an inflammatory cytokine, said method comprising the step of administering a medicament comprising an endothelial interrupter to said patient.

6. A method of improving the cognitive function of a patient suffering from a neurodegenerative disease, said method comprising the step of administering a therapeutically effective amount of an endothelial interrupter to said patient.

7. A method for treating a neurodegenerative disease in a patient in need thereof, said method comprising the step of:
administering a medicament comprising a direct thrombin inhibitor.

8. The method of claim 7, wherein said neurodegenerative disease to be treated is selected from the group consisting of: Alzheimer's disease, Parkinson's disease, Ataxia, Progressive Supranuclear Palsy, and Lewy body dementia.
